(19) United States
(12) Patent Application Publication
(10) Pub. No.: US 2012/0141323 A1
(43) Pub. Date: Jun. 7, 2012

(54) SYSTEMS AND METHODS FOR UV SANITIZATION AND STERILIZATION

(76) Inventor: Benjamin Fogg, Holland, MI (US)

(21) Appl. No.: 13/274,959

(22) Filed: Oct. 17, 2011

Related U.S. Application Data
(60) Provisional application No. 61/393,905, filed on Oct. 17, 2010.

(51) Int. Cl. A61L 2/10; G21K 5/00

(52) U.S. Cl. 422/24; 250/492.1

(57) ABSTRACT

Systems and methods for operating a UV source at maximum output are described herein.

### CROSS-REFERENCE TO RELATED APPLICATION(S)

This application claims the benefit of U.S. Provisional Application Ser. No. 61/393,905, filed Oct. 17, 2010, entitled “Systems and Methods for UV Sanitization and Sterilization,” which is hereby incorporated herein by reference in its entirety, including all references cited therein.

### BACKGROUND OF THE INVENTION

1. Field of the Invention

The present invention relates in general to systems and methods for UV sanitization and sterilization and, more particularly, but not by way of limitation, to systems and methods for effectively monitoring and operating a UV source at substantially maximum output levels.

2. Background Art

UV sanitization and sterilization apparatuses have been known in the art for years, and are the subject of numerous patents, including: U.S. Pat. No. 7,160,566 entitled “Food surface sanitation tunnel,” U.S. Pat. No. 6,911,177 entitled “Ultraviolet area sterilizer and method of area sterilization using ultraviolet radiation,” U.S. Pat. No. 6,464,760 entitled “Ultraviolet air purifier,” U.S. Pat. No. 6,348,151 entitled “Device for sterilizing and filtering water which flows through a sanitary device,” U.S. Pat. No. 5,894,130 entitled “ Ultraviolet sterilization unit,” and U.S. Pat. No. 4,156,652 entitled “Apparatus for sterilizing fluids with UV radiation and ozone”—all of which are hereby incorporated herein by reference in their entirety including the references cited therein.

U.S. Pat. No. 7,160,566 appears to disclose a modular, adjustable, easy to maintain, portable or fixed food sanitation tunnel, comprising an enclosing means for subjecting food to sanitizers including UV light, ozone, hydroperoxides, superoxides and hydroxyl radicals, and a method for using the system. The enclosing means includes one or more UV radiation sources and one or more target rods located within a tunnel, such as a c-shaped shell. The UV radiation sources are preferably UV light sources that emit UV light of approximately 185 to 254 nm. The target rods are approximately up to 0-30% titanium dioxide, up to 0-30% silver and up to 0-30% copper, by weight. The system may include a mister for the efficient production of hydroxyl radicals by the UV light sources. Parts of the system are easily removable for cleaning and for maintenance. Also, in an alternative embodiment, the tunnel is located on a frame, and the frame is on wheels.

U.S. Pat. No. 6,911,177 appears to disclose an ultraviolet area sterilizer (UVAS) that is mobile or stationary. The UVAS is positioned in a room, such as an operating room or intensive care unit. Motion detectors sense movement, to assure that personnel have evacuated the space to be sterilized. Subsequently, UV-C generators, such as mercury bulbs, generate UV-C from multiple locations within the room or other enclosed space. Multiple UV-C sensors scan the room, and determine the area reflecting the lowest level of UV-C back to the sensors. The device calculates the time required to obtain a bactericidal dose of UV-C reflected back to the sensors. Once an effective bactericidal dose has been reflected to all the sensors, the unit notifies the operator and shuts down.

U.S. Pat. No. 6,464,760 appears to disclose a portable air sterilization and filtration apparatus for removing contaminants from the ambient atmosphere, having a housing with an inlet opening and an outlet opening, filter media and ultraviolet light source, and a motorized fan for maintaining a flow of air through the housing from the inlet opening to the outlet opening. The invention also includes easy access to the filter medium and to the ultraviolet light sources for periodic replacement or cleaning, and integrates a safety lock feature whereby the removal of the filter or the removal of an ultraviolet light source would open the power circuit to the ultraviolet light source, preventing accidental irradiation of the user. The ultraviolet light sources also activate an indicator light viewable by the user when the ultraviolet light sources are energized. The invention employs a three filter media system to remove contaminants from the air stream generated within the device by the motorized fan, including a sponge filter, a HEPA type filter (high efficiency particulate air filter), which will remove 99.97% of the airborne particles of the size of 0.3 microns or larger, and an activated charcoal filter. The ultraviolet light source is disposed so as to irradiate the downstream side of the activated charcoal filter during operation of the unit to provide germicidal activity to the filter's downstream surface and to the air stream as the air stream emerges from the filter prior to its discharge through the outlet opening to return to the ambient atmosphere.

U.S. Pat. No. 6,348,151 appears to disclose a device for sterilizing and filtering water which flows through a sanitary device which consists of a treatment cavity (19) located inside of housing (2,3,4) through which water can flow. The treatment cavity is subdivided into a multitude of partial cavities 51a, 51b) by a suitably formed filter device (50). The flowing water and filter device (50) are irradiated by an ultraviolet lamp (12) and the filter device is made of a single sintered body which transmits UV radiation thereby allowing the water to be filtered to remove microorganisms and then to be killed.

U.S. Pat. No. 5,894,130 appears to disclose an ultraviolet sterilization unit having a housing attached to an air heating and cooling system. The housing including two apertures into which lamp cartridges are inserted. The lamp cartridges carrying ultraviolet lamps operating in a frequency capable of sterilizing air within the system. The cartridges are configured to automatically de-energize the lamps when a lamp cartridge is removed from the housing. When the sterilization unit is a multiple lamp system, when one of the lamp cartridges is removed all lamps are de-energized. The de-energizing of the lamps occurring before a user will view the lamp.

U.S. Pat. No. 4,156,652 appears to disclose an apparatus for sterilizing fluids comprises a radiation chamber which comprises a source of ultraviolet radiation; a housing surrounding said source and including an inner casing permeable to ultraviolet radiation and bounding a channel with said source, and an outer casing surrounding said inner casing and forming a treating space therewith; a conduit for conducting a stream of gas containing molecular oxygen through said channel for exposure to said ultraviolet radiation to produce an ozone-enriched gas; a conduit for conducting a fluid through said treating space so as to become sterilized by the ultraviolet radiation; and a conduit for introducing at least a portion of said ozone-enriched gas from said channel into said treating space to become united with said fluid, whereby said introduced portion of ozone-enriched gas is again exposed to ultraviolet radiation in order to produce an increased content of ozone in said ozone-enriched gas and an additional sterilizing effect is produced in said fluid. A process for sterilizing fluid is also provided. According to this process, a fluid and a gas which contains molecular oxygen are irradiated with ultraviolet radiation and are then mixed together in order to produce an additional sterilizing effect.

While the above-identified patents and publications do appear to provide UV sanitization and/or sterilization apparatuses, their configurations remain non-desirous and/or problematic inasmuch as, among other things, none of the above-identified apparatuses appear to be configured to effectively monitor and maintain a UV source at substantially maximum output levels—among other things.

It is therefore an object of the present invention to provide systems and methods, which, among other things, remedy the aforementioned detriments and/or complications associated with the use of the above-identified, conventional UV sanitation and/or sterilization apparatuses, in particular effectively monitoring and maintaining a UV source at substantially maximum output levels.

These and other objects of the present invention will become apparent in light of the present specification, claims, and drawings.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be further understood that the invention is not necessarily limited to the particular embodiments illustrated herein.

The invention will now be described with reference to the drawings wherein:

FIG. 1 of the drawings is an exemplary environment for practicing the present invention;

FIG. 2 of the drawings is a block diagram of a control module for practicing embodiments of the present invention; and

FIG. 3 of the drawings is a block diagram of an exemplary computing system for executing one or more functions of a method for maintaining a UV source at maximum output, in accordance with various embodiments of the present invention.

### DETAILED DESCRIPTION OF THE INVENTION

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings with like reference characters.

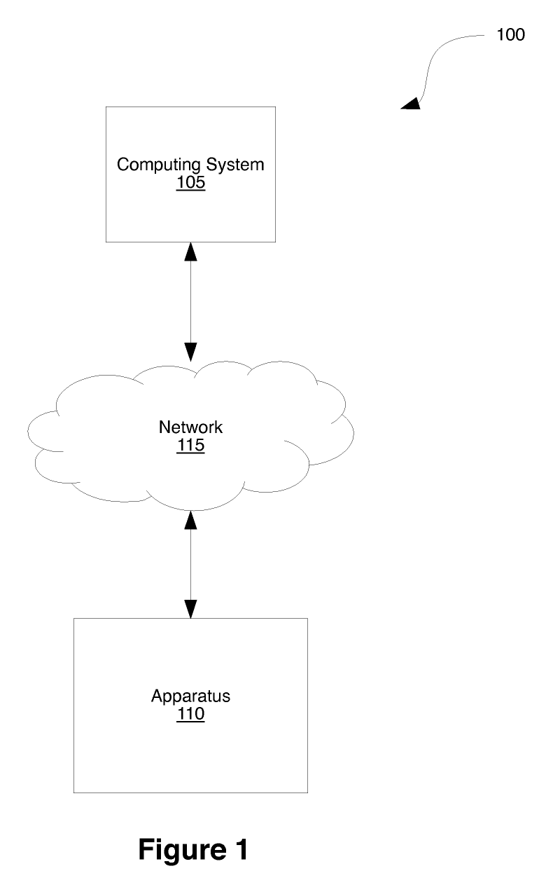

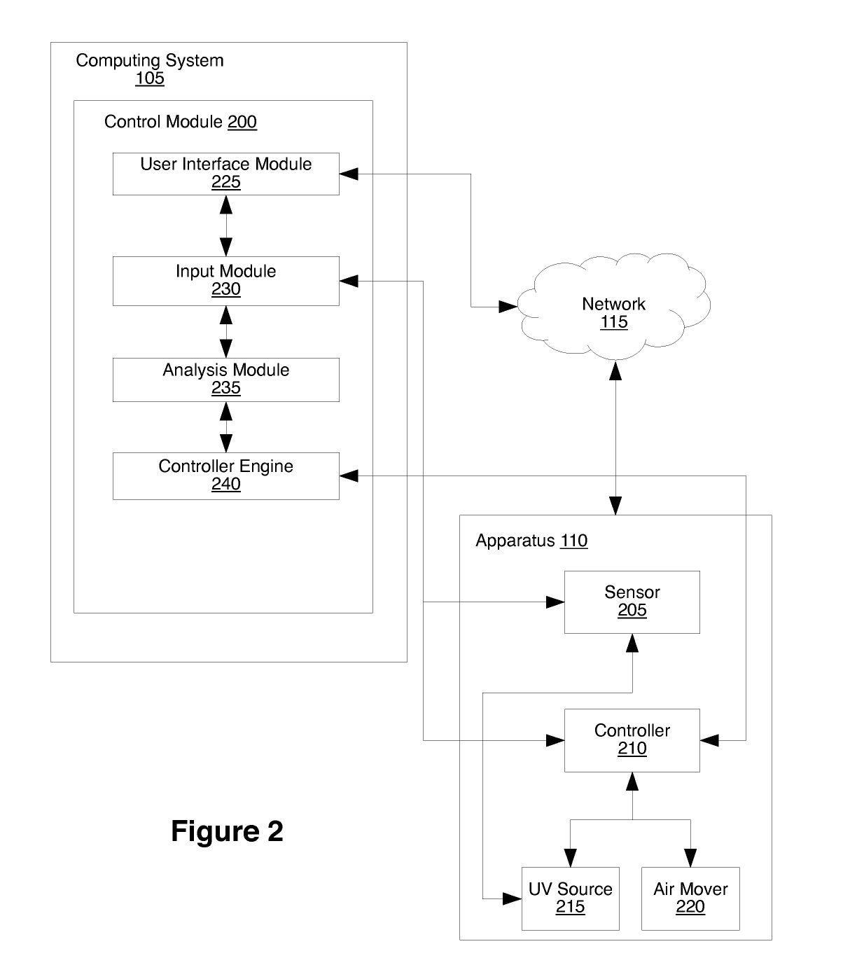

Referring now to the collective drawings (i.e., FIGS. 1-4), shown therein is an exemplary environment 100 for practicing the present invention. In one embodiment, environment 100 includes computing system 105 adapted to control UV sanitization and sterilization apparatus 110. According to some embodiments, computing system 105 is operatively connected to apparatus 110 via network 115. It will be understood that network 115 may include a LAN (Local Area Network), a WAN (Wide Area Network), a VPN tunnel (Virtual Private Network), or other connection such as a direct port such as Ethernet, firewire, USB (Universal Serial Bus), or similar ports.

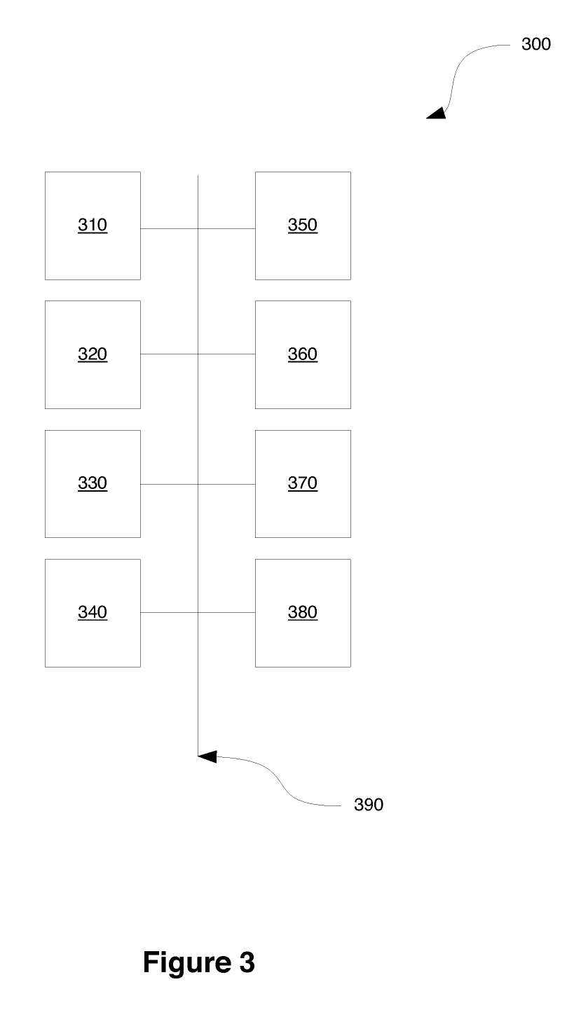

Exemplary descriptions of computing system 105 contemplated for use in accordance with the present invention are provided with respect to computing system 300 shown in FIG. 3, described in greater detail infra. According to some embodiments, apparatus 110 may include any apparatus, device, or assembly adapted to sanitize and/or sterilize beverage container components such as bottle caps, although one of ordinary skill in the art will readily appreciate that many other types of objects may be sanitized or sterilized by apparatus 110.

It will be understood that apparatus 110 is preferably capable of between 2 and 5+ Log reduction in the amount of “undesirable matter” present on objects communicating through apparatus 110. In particular, operating apparatus 110 at a first temperature and for a predetermined period of time causes between 2 and 4 Log reductions (sanitization) in the amount of undesirable matter on the objects. Additionally, operating apparatus 10 at either: (1) a temperature greater than the first temperature; or (2) for a longer period of time will result in a 5+ Log reduction (sterilization) in the amount of undesirable matter on the objects. It will be further understood that the term “undesirable matter” includes for example, microorganisms, bacteria, fungi, and/or any other neutralizable matter that is deemed unacceptable on or within any part of an object utilized in the food and/or beverage industries, or other industries such as the medical device, computer component, or other similar industries.

Generally speaking, regardless of the configuration, apparatus 110 may be operatively associated with control module 200 which is, in turn, operatively associated with one or more of sensor 205 and controller 210 that modify the operations of at least one of UV source 215 and air mover 220 associated with apparatus 110.

Control module 200 may be adapted to maintain UV source 215 at maximum output levels and cause efficient reductions of undesirable matter by selectively controlling the output of UV source 215 by monitoring, evaluating, and varying both the flow of air within apparatus 110 and the UV-C output of UV source 215. The above-described functionalities may be facilitated by one or more modules or engines of control module 200 such as user interface module 225, input module 230, analysis module 235, and controller engine 240. It will be understood that one or more of the modules or engines of control module 200 may reside on either computing system 105 or apparatus 110. It is noteworthy that control module 200 may be composed of more or fewer modules and engines (or combinations of the same) and still fall within the scope of the present technology. Additionally, it will be understood that the constituent modules described herein may be executed by a processor of a computing system (see FIG. 3) to effectuate respective functionalities attributed thereto.

In some embodiments, UV source 215 may include a plurality of elongated UV bulbs capable of producing at least one of UV-A, B, and C light. Non-limiting suitable examples of UV bulbs for use in apparatus 110 include any commercially available non-xenon germicidal UV bulbs available from such companies as Osram Sylvania and Phillips Global, although other UV bulbs that would be known to one of ordinary skill in the art with the present disclosure before them are likewise contemplated for use in accordance with the present invention.

In one embodiment, sensor 205 includes a photospectrometer capable of sensing UV light, and particularly the UV-C light emitted by UV source 215. It will be understood that apparatus 110 may include one or more sensors 205 capable of sensing other types of electromagnetic radiation. Sensor 205 is capable of sensing the amount of UV-C light output by UV source 215 and outputting signals indicative of the same to input module 230.

In general, UV source 215 has declining temperature profiles such that when initially energized the UV-C light output of UV source 215 reach a maximum output level and thereafter decline to an output level that is substantially constant, but sometimes lower than maximum output level. This phenomenon may cause deleterious reductions the sanitizing and/or sterilizing capabilities of UV source 215 as compared to UV sources 215 operating at substantially maximum output.

Air mover 220 may be any number of devices capable of delivering a continuous flow of air into apparatus 110 such as a fan, blower, or the like. Air mover 220 may be operatively connected to controller 210 that in turn receives signals from controller engine 240.

User interface module 225 may be adapted to generate one or more user interfaces that allow end users to interact with control module 200 to establish and modify settings that control apparatus 110. Input module 230 is adapted to receive input from at least one of sensor 205, controller 210, or from a user interface generated by the user interface module 225. The signals from sensor 205 and controller 210 may be received by input module 230 at predetermined intervals, or automatically and continuously.

Information received by input module 230 may be communicated to analysis module 235 that may include one or more algorithms adapted to cause UV source 215 to operate at sustained maximum output levels. More specifically, analysis module 235 may be adapted to determine a maximum operational output of UV source 215 by activation of sensor 205 via controller engine 240. Sensor 205 is adapted to measure the UV-C output of UV source 215, which may be activated by controller 210 via controller engine 240. As sensor 205 receives data indicative of the UV-C produced by UV source 215, sensor 205 outputs data indicative of the same input module 230. Analysis module 235 evaluates sensor 205 outputs received by input module 230 to determine the actual maximum output of UV source 215.

Once analysis module 235 determines the maximum actual output of UV source 215, analysis module 235 selectively controls the operation of air mover 220 and UV source 215 through controller 210 via controller engine 240 to maintain UV source 215 at a substantially maximum output level.

Typically, the only way to determine if one or more of the UV bulbs of UV source 215 are underperforming is to detect measurable increases in the amount of undesirable material present on objects sanitized or sterilized by apparatus 110. Unfortunately, by the time undesirable materials can be measured, contamination may be widespread, affecting many or all of the objects sanitized or sterilized by apparatus 110. Therefore, analysis module 235 may be adapted to determine if one or more of the UV bulbs of UV source 215 are underperforming by receiving output from sensor 205 and comparing known maximum output levels of UV source 215 to actual output levels measured by sensor 205. Declining temperature profiles for UV source 215 may be indicative of one or more underperforming UV bulbs.

According to other embodiments, a database may be utilized by control module 200 to record and to notify operators of various data relative to UV source 215 performance such as output levels, temperature, and the like. The data collected may be organized into logs that can be stored in records that may be indexed and accessed by user interface module 225. More specifically, user interface module 225 may be adapted to generate visual displays corresponding to analytics relative to the operation of UV source 215, including log data indicative of UV-C output levels, temperature, and the like.

FIG. 3 illustrates an exemplary computing system 300 that may be used to implement various portions of the present invention. Computing system 300 of FIG. 3 may be implemented in the context of computing system 105, control module 200, and the like. Computing system 300 of FIG. 3 includes one or more processors 310 and main memory 320. Main memory 320 stores, in part, instructions and data for execution by processor 310. Main memory 320 can store the executable code when computing system 300 is in operation. Computing system 300 of FIG. 3 may further include mass storage device 330, portable storage medium drive(s) 340, output devices 350, user input devices 360, graphics display 370, and other peripheral devices 380.

The components shown in FIG. 3 are depicted as being connected via single bus 390. The components may be connected through one or more data transport means. Processor 310 and main memory 320 may be connected via a local microprocessor bus, and mass storage device 330, peripheral device(s) 380, portable storage medium drive 340, and graphics display 370 may be connected via one or more input/output (I/O) buses.

Mass storage device 330, which may be implemented with a magnetic disk drive or an optical disk drive, is a non-volatile storage device for storing data and instructions for use by processor 310. Mass storage device 330 can store the system software for implementing embodiments of the present invention for purposes of loading that software into main memory 320.

Portable storage medium drive 340 operates in conjunction with a portable non-volatile storage medium, such as a floppy disk, compact disk or Digital video disc, to input and output data and code to and from computing system 300 of FIG. 3. The system software for implementing embodiments of the present invention may be stored on such a portable medium and input into computing system 300 via portable storage medium drive 340.

Use input devices 360 provide a portion of a user interface. User input devices 360 may include an alphanumeric keypad, such as a keyboard, for inputting alphanumeric and other information, or a pointing device, such as a mouse, a trackball, stylus, or cursor direction keys. Additionally, computing system 300 as shown in FIG. 3 includes output devices 350. Suitable output devices include speakers, printers, network interfaces, and monitors.

Graphics display 370 may include a liquid crystal display (LCD) or other suitable display device. Graphics display 370 receives textual and graphical information, and processes the information for output to the display device.

Peripheral devices 380 may include any type of computer support device to add additional functionality to the computer system. Peripheral device(s) 380 may include a modem or a router.

The components contained in computing system 300 of FIG. 3 are those typically found in computer systems that may be suitable for use with embodiments of the present invention and are intended to represent a broad category of such computer components that are well known in the art. Thus, computing system 300 of FIG. 3 can be a personal computer, hand held computing system, telephone, automated bank teller machine (ATM), mobile computing system, workstation, server, minicomputer, mainframe computer, or any other computing system. The computer can also include different bus configurations, networked platforms, multi-processor platforms, etc. Various operating systems can be used including UNIX, Linux, Windows, Macintosh OS, Palm OS, iOs, and other suitable operating systems.

Some of the above-described functions may be composed of instructions that are stored on storage media (e.g., computer-readable medium). The instructions may be retrieved and executed by the processor. Some examples of storage media are memory devices, tapes, disks, and the like. The instructions are operational when executed by the processor to direct the processor to operate in accord with the invention. Those skilled in the art are familiar with instructions, processor(s), and storage media.

It is noteworthy that any hardware platform suitable for performing the processing described herein is suitable for use with the invention. The terms “computer-readable storage medium” and “computer-readable storage media” as used herein refer to any medium or media that participate in providing instructions to a CPU for execution. Such media can take many forms, including, but not limited to, non-volatile media, volatile media and transmission media. Non-volatile media include, for example, optical or magnetic disks, such as a fixed disk. Volatile media include dynamic memory, such as system RAM. Transmission media include coaxial cables, copper wire and fiber optics, among others, including the wires that comprise one embodiment of a bus. Transmission media can also take the form of acoustic or light waves, such as those generated during radio frequency (RF) and infrared (IR) data communications. Common forms of computer-readable media include, for example, a floppy disk, a flexible disk, a hard disk, magnetic tape, any other magnetic medium, a CD-ROM disk, digital video disk (DVD), any other optical medium, any other physical medium with patterns of marks or holes, a RAM, a PROM, an EPROM, an EEPROM, a FLASHEPROM, any other memory chip or cartridge, a carrier wave, or any other medium from which a computer can read.

Various forms of computer-readable media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU.

While the present invention has been described in connection with a series of preferred embodiments, these descriptions are not intended to limit the scope of the invention to the particular forms set forth herein. It will be further understood that the methods of the invention are not necessarily limited to the discrete steps or the order of the steps described. To the contrary, the present descriptions are intended to cover such alternatives, modifications, and equivalents as may be included within the spirit and scope of the invention as defined by the appended claims and otherwise appreciated by one of ordinary skill in the art.

What is claimed and desired to be secured by Letters Patent of the United States is:

1. A system for operating a UV source at maximum output as provided in FIGS. 1-3 having one or more of the disclosed structural, functional, and/or ornamental characteristics.

2. A method for operating a UV source at maximum output in combination with an ultraviolet sanitation and sterilization apparatus as provided herein having one or more of the disclosed structural, functional, and/or ornamental characteristics.
